(19) United States
(12) Patent Application Publication
(10) Pub. No.: US 2023/0347149 A1
(43) Pub. Date: Nov. 2, 2023

(54) METHOD OF ELECTROCHEMICAL SEPARATION OF MEMBRANES OF VIRUSES WITH LIPIDS THAT CAUSE INFECTION IN HUMAN AND ANIMALS

(71) Applicants: Serkan TUNC, Istanbul (TR); Sevil TUNC, Istanbul (TR)

(72) Inventors: Serkan TUNC, Istanbul (TR); Sevil TUNC, Istanbul (TR)

(73) Assignees: Serkan TUNC, Istanbul (TR); Sevil TUNC, Istanbul (TR)

(21) Appl. No.: 17/791,245

(22) PCT Filed: Aug. 16, 2021
(86) PCT No.: PCT/TR2021/050815

§ 371 Date: Jul. 7, 2022

(30) Foreign Application Priority Data
Sep. 17, 2020 (TR) 2020/14781

(51) Int. Cl. A61N 1/36

(52) U.S. Cl. CPC A61N 1/3606

(57) ABSTRACT

The invention relates to the dissociation of molecular bonds of water (H₂O), sodium chloride (NaCl) and potassium chloride (KCl) salts in tissue fluids and blood, using the energy created by connecting human and animal bodies in series with the electrical circuit to act as a resistor and thereby enabling the formation of sodium hydroxide (NaOH) and potassium hydroxide (KOH) lipid-solvents for the decomposition of the lipid layer that forms the membranes of viruses in tissue fluids and blood in human and animal bodies. This method prevents viruses with lipid membranes from infecting the human and animal body after transmission, and neutralizes viruses with lipid membranes in blood and tissue fluid by separating these membranes.

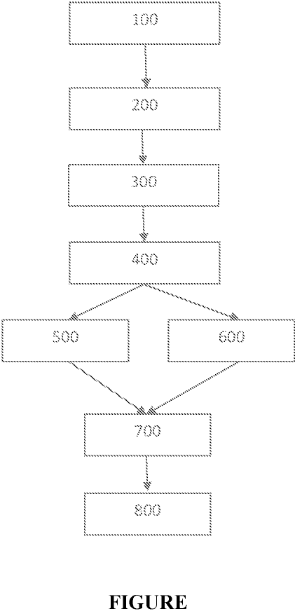

### CROSS REFERENCE TO THE RELATED APPLICATIONS

This application is the national phase entry of International Application No. PCT/TR2021/050815, filed on Aug. 16, 2021, which is based upon and claims priority to Turkish Patent Application No. 2020/14781, filed on Sep. 17, 2020, the entire contents of which are incorporated herein by reference.

### TECHNICAL FIELD

The invention relates to a method of electrochemical separation of the membranes of viruses with lipid membranes that cause infection in tissue fluids and blood of human and animal bodies.

### BACKGROUND

In the state of the art, immunity after infections caused by viruses with lipid membranes in human and animal blood occurs when the body defeats the infection after treatment with vaccines and drugs. However, the development and administration of vaccines, which is one of the most important methods used in treatment, takes a long time, which does not make it possible to use the vaccine in emergency situations and creates the need for other treatment methods.

One of the most important methods used in treatment is drugs. However, not knowing what kind of side effects will be caused by drugs used in new types of diseases poses a great risk.

The intended chemical changes in the human and animal body are initiated by the direct transmission of various chemicals to the body as solid, liquid and gas. The effect of another chemical is used to initiate a chemical reaction in the body. In this case, the side effects of the chemicals in question have to be tolerated and unexpected results may be encountered.

### SUMMARY

The invention relates to the dissociation of molecular bonds of water (H₂O), sodium chloride (NaCl) and potassium chloride (KCl) salts in tissue fluids and blood, using the energy created by connecting human and animal bodies in series with the electrical circuit to act as a resistor and thereby enabling the formation of sodium hydroxide (NaOH) and potassium hydroxide (KOH) lipid-solvents for the decomposition of the lipid layer that forms the membranes of viruses in tissue fluids and blood in human and animal bodies. This method prevents viruses with lipid membranes from infecting the human and animal body after transmission, and neutralizes viruses with lipid membranes in blood and tissue fluid by separating these membranes.

### BRIEF DESCRIPTION OF THE DRAWINGS

The FIGURE describing the method of electrochemical separation of the membranes of viruses with lipid membranes that cause infection in humans and animals is shown below.

The FIGURE: Flow chart of the electrochemical separation of the membranes of viruses with lipid membranes that cause infections in humans and animals.

### EXPLANATION OF THE REFERENCES IN FIGURE:

The corresponding reference numbers shown on the flow chart for the method of electrochemical separation of the membranes of viruses with lipid membranes that cause infection in humans and animals are given below.
100. Generating electric current with direct current converted by a direct current generator from a circuit embodiment formed with a direct current electricity generator, an electrical frequency generator, a human or animal body that acts as a resistor, a switch that works on and off and coupling of said elements in series with each other with a conductor wire,
200. Generating electrical frequency with electrical frequency generator and obtaining direct current with frequency that can be adjusted according to the desired chemical reaction,
300. Ensuring the occurrence of chemical reactions in the tissue fluid and blood compounds in the human or animal body with the energy formed inside the human or animal body by passing the current through the human or animal body used as a resistor,
400. Decomposition of water (H₂O), as hydrogen (H⁺) and hydroxide (OH⁻) in tissue fluid and blood in the human or animal body; decomposition of sodium chloride (NaCl) as sodium (Na⁺) and chloride (Cl-); decomposition of potassium chloride (KCl), as potassium (K⁺) and chloride (Cl⁻) with the energy created inside the human or animal body and thus eliminating the ion deficiency in the human and animal body,
500. Ensuring that the sodium (Na⁺) and hydroxide (OH⁻) formed in the tissue fluid and blood in the human or animal body form a bond together with the energy created in the human or animal body, and thereby forming sodium hydroxide (NaOH),
600. Ensuring that the sodium (K⁺) and hydroxide (OH⁻) formed in the tissue fluid and blood in the human or animal body form a bond together with the energy created in the human or animal body, and thereby forming potassium hydroxide (KOH),
700. Continuing the process of forming potassium hydroxide (KOH) and sodium hydroxide (NaOH) until the lipid layer, which forms the membranes of viruses in tissue fluids and blood of human and animal bodies, decomposes,
800. Potassium hydroxide (KOH) and sodium hydroxide (NaOH), which are formed in sufficient amounts in the human and animal body, showing a saponification reaction by chemically reacting with the lipid membranes of the viruses, thereby causing separation of the membrane and the destruction of the virus’s integrity.

### DETAILED DESCRIPTION OF THE EMBODIMENTS

The invention relates to an electrical circuit created by making use of the ability of human and animal bodies to pass electric current and resist this current, and to create chemical reactions in tissue fluids and blood in human and animal bodies with the energy obtained thanks to this electric circuit .

The circuit in which the human and animal body is used to act as a resistor has an embodiment which is composed of a direct current generator that converts alternating current to direct current, an electrical frequency generator, a human/animal body that acts as a resistor, a switch operating on and off, and coupling of the said elements in series with each other with a conductive wire.

In the circuit created to achieve the aim of the invention, there is a direct current electricity generator that converts alternating current to direct current. In order to generate frequency in direct current, an electrical frequency generator has been integrated into the DC electricity generator by serial connection.

There is at least one switch that turns the circuit on and off in order to control the flow of current in the said circuit. The human or animal body is connected in series with the circuit as a resistor in order to generate a current in the circuit. In the circuit in question, in order to obtain higher efficiency from the human or animal body by using a lower electrical voltage, frequency generator that provides frequency characteristic to the current coming out of the direct current generator and is connected to the circuit in series, the direct current was added to the circuit by connecting to the output of the electricity generator.

When the energy level exceeding the molecular bond dissociation energy levels of water (H₂O), sodium chloride (NaCl) and potassium chloride (KCl) salts in the body is reached by giving current to the circuit in question, water (H₂O) decomposes into hydrogen (H⁺) and hydroxide (OH⁻); sodium chloride (NaCl) decomposes into sodium (Na⁺) and chloride (Cl⁻), and potassium chloride (KCl) decomposes into potassium (K⁺) and chloride (Cl⁻).

It is ensured that the sodium (Na⁺) and hydroxide (OH⁻) formed in the tissue fluid and blood in the human or animal body form a bond together with the energy created in the human or animal body, and thereby forming sodium hydroxide (NaOH). It is also ensured that the sodium (K⁺) and hydroxide (OH⁻) formed in the tissue fluid and blood in the human or animal body form a bond together with the energy created in the human or animal body, and thereby forming potassium hydroxide (KOH).

Until the lipid layer, which forms the membranes of viruses in the tissue fluids and blood of human and animal bodies, separates, the process of forming potassium hydroxide (KOH) and sodium hydroxide (NaOH) continues in the body. In other words, the body continues to receive current.

Potassium hydroxide (KOH) and sodium hydroxide (NaOH), which are formed in sufficient quantities in human and animal bodies, enter into a chemical reaction with the lipid membranes of the viruses and creates a saponification reaction, causing the membrane to decompose and the integrity of the virus to deteriorate. Thus, viruses in tissue fluids and blood in human and animal bodies are rendered ineffective.

The electrochemical separation method of lipid membrane viruses that cause infections in humans and animals consists of the following steps:
generating electric current with direct current converted by a direct current generator from a circuit embodiment formed with a direct current electricity generator, an electrical frequency generator, a human or animal body that acts as a resistor, a switch that works on and off and coupling of said elements in series with each other with a conductor wire (100),
generating electrical frequency with electrical frequency generator and obtaining direct current with frequency that can be adjusted according to the desired chemical reaction (200),
ensuring the occurrence of chemical reactions in the tissue fluid and blood compounds in the human or animal body with the energy formed inside the human or animal body by passing the current through the human or animal body used as a resistor (300),
decomposition of water (H₂O), as hydrogen (H⁺) and hydroxide (OH⁻) in tissue fluid and blood in the human or animal body; decomposition of sodium chloride (NaCl) as sodium (Na⁺) and chloride (Cl⁻); decomposition of potassium chloride (KCl), as potassium (K⁺) and chloride (Cl⁻) with the energy created inside the human or animal body and thus eliminating the ion deficiency in the human and animal body (400),
ensuring that the sodium (Na⁺) and hydroxide (OH⁻) formed in the tissue fluid and blood in the human or animal body form a bond together with the energy created in the human or animal body, and thereby forming sodium hydroxide (NaOH) (500),
ensuring that the sodium (K⁺) and hydroxide (OH⁻) formed in the tissue fluid and blood in the human or animal body form a bond together with the energy created in the human or animal body, and thereby forming potassium hydroxide (KOH) (600),
continuing the process of forming potassium hydroxide (KOH) and sodium hydroxide (NaOH) until the lipid layer, which forms the membranes of viruses in tissue fluids and blood of human and animal bodies, decomposes (700),
potassium hydroxide (KOH) and sodium hydroxide (NaOH), which are formed in sufficient amounts in the human and animal body, showing a saponification reaction by chemically reacting with the lipid membranes of the viruses, thereby causing separation of the membrane and the destruction of the virus’s integrity (800).

### INDUSTRIAL APPLICATIONS OF THE INVENTION

The invention is a method used for forming various chemicals in tissue fluids and blood of human and animal body and is easily applicable to the industry.

What is claimed is:

1. An electrochemical separation method of lipid membrane of viruses that cause infections in humans and animals, comprising the following steps:
generating an electric current with direct current converted by a direct current generator from a circuit embodiment formed with elements comprising a direct current electricity generator, an electrical frequency generator, a human or animal body acting as a resistor, a switch for turning on and off and coupling of the elements with a conductor wire, wherein the elements are in series with each other,
generating electrical frequency with the electrical frequency generator and obtaining the direct current with a frequency, wherein the frequency is adjustable according to a desired chemical reaction,
ensuring an occurrence of the desired chemical reaction in tissue fluid and blood in the human or animal body with an energy formed inside the human or animal body by passing the electric current through the human or animal body,
decomposition of water (H₂O) as hydrogen (H⁺) and hydroxide (OH⁻) in the tissue fluid and the blood in the human or animal body; decomposition of sodium chloride (NaCl) as sodium (Na⁺) and chloride (Cl⁻); decomposition of potassium chloride (KCl), as potassium (K⁺) and chloride (Cl⁻) with the energy created inside the human or animal body and thus eliminating an ion deficiency in the human and animal body,
ensuring that the sodium (Na⁺) and hydroxide (OH⁻) formed in the tissue fluid and the blood in the human or animal body form a bond together with the energy created in the human or animal body, and thereby forming sodium hydroxide (NaOH),
ensuring that the sodium (K⁺) and hydroxide (OH⁻) formed in the tissue fluid and the blood in the human or animal body form a bond together with the energy created in the human or animal body, and thereby forming potassium hydroxide (KOH),
continuing the process of forming the potassium hydroxide (KOH) and the sodium hydroxide (NaOH) until a lipid layer, forming the lipid membranes of the viruses in the tissue fluid and the blood of the human and animal body decomposes,
allowing the potassium hydroxide (KOH) and the sodium hydroxide (NaOH), to be formed in sufficient amounts in the human and animal body for a saponification reaction, wherein the potassium hydroxide (KOH) and the sodium hydroxide (NaOH) chemically react with the lipid membranes of the viruses, thereby causing separation of the lipid membranes and destruction of integrity of the viruses .
